(19) United States
(12) Patent Application Publication
(10) Pub. No.: US 2017/0266138 A1
(43) Pub. Date: Sep. 21, 2017

(54) METHODS OF TREATMENT OF ACNE VULGARIS USING TOPICAL DAPSONE COMPOSITIONS

(71) Applicant: Allergan, Inc., Irvine, CA (US)

(72) Inventors: Kevin S. Warner, Anaheim, CA (US); Ajay P. Parashar, Fairfax, VA (US); Vijaya Swaminathan, Bangalore (IN); Varsha Bhatt, San Francisco, CA (US); Alexandre Kaoukhov, Newport Beach, CA (US)

(21) Appl. No.: 15/611,551

(22) Filed: Jun. 1, 2017

Related U.S. Application Data
(63) Continuation of application No. 15/343,978, filed on Nov. 4, 2016.
(60) Provisional application No. 62/250,763, filed on Nov. 4, 2015.
(60) Provisional application No. 62/299,978, filed on Feb. 25, 2016.

(51) Int. Cl. A61K 31/145; A61K 47/32; A61K 47/10; A61K 47/14

(52) U.S. Cl. CPC A61K 31/145; A61K 47/14; A61K 47/32; A61K 47/10

(57) ABSTRACT

Dapsone compositions can be useful for treating acne. The methods and formulations disclosed herein show efficacy for treating acne vulgaris and/or post inflammatory hyperpigmentation.

### CROSS-REFERENCE TO RELATED APPLICATIONS

This application is a continuation of U.S. patent application Ser. No. 15/343,978, filed on Nov. 4, 2016, which claims the benefit of U.S. Provisional Application No. 62/250,763, filed on Nov. 4, 2015, and U.S. Provisional Application No. 62/299,978, filed on Feb. 25, 2016, the entire content of all applications are incorporated herein by reference.

### FIELD

The present embodiments relate generally to methods of treatment of acne vulgaris and/or post-inflammatory hyperpigmentation with topical dapsone compositions.

### BACKGROUND

Acne is a group of common skin conditions characterized by the so-called “acneiform” or acne-like skin eruptions, which can be contaminated with bacteria, such as Propionibacterium acnes, and can also be marked by inflammation. Acne tends to occur in the areas of skin where the sebaceous glands are most active, such as the face. Acne is associated with psychological trauma, and, if left untreated, can lead to scar formation and disfigurement.

Classification and the diagnosis of various acne conditions can be complex, and even contradictory. Given this complexity and unpredictability, medication and other therapies, are often developed on a trial-and-error basis in order to determine the most effective course of treatment for a particular patient. The outcome of any particular acne treatment regimen greatly varies from patient to patient, as well as throughout treatment of a particular patient. In addition to the complexity and variability of acne conditions, treatment efficacy can be greatly affected by a patient's compliance with the treatment regimen. Patient compliance during acne treatment may be influenced by side effects, which, for topical medications, commonly include redness, itching, and skin peeling. The complexity of the drug regimen can also negatively affect patient compliance, particularly where two or more different topical medications are prescribed simultaneously. Another factor that negatively affects patient compliance is the cost of a drug regiment, which is considerably higher when multiple medications are prescribed. In some countries, acne is considered a cosmetic problem, and acne treatments are not covered by insurance plans, thus further increasing patient's treatment costs. Certain compositions for treatment of acne are available. Many of the available compositions include one active agent known to have anti-acne activity. Stability of compositions with multiple anti-acne agents can be problematic. Also, these compositions can be difficult to manufacture.

Accordingly, there is a continuing need for compositions and methods used in a treatment of acne, in which topical application is potentially effective. The compositions and methods provided herein address these and other needs in the art.

### SUMMARY

Dapsone, (4,4′-diaminodiphenyl sulfone) is a medicament possessing several beneficial medicinal activities. Dapsone is typically administered as one of the medicinal agents used in the treatment of leprosy. Dapsone and its derivatives are also effective for treatment of bacterial infections, protozoal infections such as malaria, pneumocystis carinii, and plasmonic infections such as toxoplasmosis.

Dapsone is also useful as an anti-inflammatory agent. It has been used to treat skin diseases characterized by the abnormal infiltration of neutrophils, such as Dermatitis herpetiformis, linear IgA dermatosis, pustular psoriasis, pyoderma gangrenosum, acne vulgaris, and Sweet's Syndrome. Examples of dapsone formulations useful in the present application are found in U.S. Pat. No. 9,161,926, which is herein incorporated by reference in its entirety.

Use of topical compositions of dapsone can be problematic. Topical compositions may act as drying agents for the skin. They remove essential oils and natural skin softeners from the skin thus causing it to be dry, itch and crack. Inclusion of exogeneous skin emollients, oils and the like, however, causes phase separation and precipitation of dapsone. Use of typical emulsifiers does not solve the dapsone precipitation owing to the lowered dapsone solubility and conflicting physical characteristics of the phases of the resulting composition. in particular, topical compositions including dapsone and methods are needed that would, for example, exhibit improved effectiveness, reduced side effects, or both, When used in a particular patient with a skin condition. Such improved topical compositions including dapsone and methods of their uses are also needed to improve treatment of patients with acne or suspected acne. The present methods using dapsone formulations can be useful for treating a variety of dermatological conditions. Some useful compositions include dapsone in a polymeric viscosity builder. Some compositions can be adjusted to optimize the dermal delivery profile of dapsone to effectively treat dermatological conditions and improve the efficiency of pharmaceutical products applied to the skin. Diethylene glycol monoethyl ether is a solubilizer for dapsone, thereby allowing compositions to be prepared with increased solubilized concentrations of dapsone. As a result, the compositions described herein are effective in treating dermatological conditions in a subject in need thereof.

In one embodiment, there are provided compositions including dapsone, a first solubilizing agent which is diethylene glycol monoethyl ether, optionally at least one second solubilizing agent, a polymeric viscosity builder, and water, wherein the dapsone is present at a concentration of about 5% w/w to about 10% w/w.

In one embodiment, there are provided compositions including dapsone, a first solubilizing agent which is diethylene glycol monoethyl ether, optionally at least one second solubilizing agent, a polymeric viscosity builder, and water, wherein the dapsone is present at a concentration of about 3% w/w to 8% w/w.

In another embodiment, there are provided methods for treating a dermatological condition. Such methods can be performed, for example, by administering to a subject in need thereof a therapeutically effective amount of a pharmaceutical composition described herein.

In some embodiments, there are provided methods for treating acne vulgaris by administering a 7.5% w/w dapsone formulation at a frequency of once a day. In some embodiments, the methods significantly reduce lesion count in a period of time in the range of two weeks to twelve weeks. In some embodiments, the incidences of adverse events, such as erythema, scaling, dryness, and/or stinging/burning decrease over treatment, In some embodiments the methods result in very few instances (e.g. <1%) of redness, dryness, and peeling of treated skin.

In sonic embodiments, a method of treating acne vulgaris in a subject in need thereof, includes administering a topical pharmaceutical composition comprising about 7.5% w/w dapsone to the entire face of the subject at a frequency of once a day for a treatment duration effective to improve the acne vulgaris. The treatment duration can be in the range of about 4 weeks to about 12 weeks. The method can be therapeutically effective to reduce the number of lesions on the face of the subject. In some embodiments, the topical pharmaceutical composition does not comprise adapalene. According to an embodiment, the treatment duration is 12 weeks, In some embodiments, the lesions are inflammatory lesions. In some embodiments, the lesions are non-inflammatory lesions. According to some embodiments, the method is effective to reduce the amount of local cutaneous irritation in the subject over the treatment duration.

In some embodiments, the local cutaneous irritation comprises erythema. In some embodiments, the local cutaneous irritation comprises scaling. According to some embodiments, the local cutaneous irritation comprises dryness. In some embodiments, the local cutaneous irritation comprises stinging/burning. In some embodiments, the topical pharmaceutical composition further comprises about 30% w/w diethylene glycol monoethyl ether. In some embodiments, the topical pharmaceutical composition further comprises 4% w/w of a polymeric viscosity builder consisting of acrylamide/sodium acryloyldimethyl taurate copolymer. In some embodiments, the topical pharmaceutical composition further comprises methyl paraben.

### BRIEF DESCRIPTION OF THE FIGURES

FIG. 1 illustrates the mean percent reduction of acne lesions from baseline over time when comparing formulations of the invention to vehicle, when administered at a frequency of once a day.

FIG. 2 is a chart illustrating the local cutaneous irritation profile over time (at baseline, at maximum severity, and at the end of a 12 week treatment regimen) when formulations of the invention were administered at a frequency of once a day to treat acne vulgaris.

### DETAILED DESCRIPTION

It is to be understood that both the foregoing general description and the following detailed description are exemplary and explanatory only and do not restrict the claims. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

Some embodiments include compositions and products for treatment of skin conditions and methods of treating skin conditions. The term “skin condition” as used herein encompasses human and animal conditions, disorders, or diseases affecting skin. Such skin conditions include, but are not limited to, conditions involving skin inflammation, conditions involving sebaceous glands and hair follicles, conditions characterized by acneiform symptoms, and conditions involving skin dryness, skin thickening, skin scaling or skin flaking. Skin conditions that can be treated using some compositions, products and methods described herein include, but are not limited to, acne, rosacea, folliculitis, perioral dermatitis, photodamage, skin aging, psoriasis, ichtiosis, atopic dermatitis, treatment of chronic wounds, bed sores, keratosis piralis, scars, including surgical and acne scars, sebaceous cysts, inflammatory dermatoses, post inflammatory hyperpigmentation, eczema, xerosis, pruritis, lichen planus, nodular prurigo, eczema, and miliaria.

The term “acne” as used herein, encompasses skin conditions involving acneiform or acne-like symptoms. For example, a skin condition characterized by follicular eruptions, such as papules and pustules resembling acne, can be categorized as acne. It is to be understood that the term “acne” is not to be limited to diseases and conditions characterized by papules and pustules, but can be characterized by a variety of symptoms. It is also to be understood that a particular patient having acne can be in remission, or the patient's acne can be controlled by continuing treatments, and therefore the patient can exhibit reduced symptoms or be asymptomatic. Nevertheless, continuing treatment of acne can be recommended in such a patient in order to reduce the probability of the return of the acne symptoms.

Symptoms of acne or acne-like conditions include, but are not limited to, the appearance of various skin lesions. The term “lesion” is generally used to denote an infected or diseased patch of skin. A lesion can involve an infected sebaceous gland. Some lesions are more severe than others. Examples of skin lesions are comedones, macules, papules, pustules, nodules and cysts. The term “comedo” (plural “comedones”) is used to describe a sebaceous follicle plugged with dirt, other cells, tiny hairs, or bacteria. Comedones include the so-called “blackheads,” which can also refer to as “open comedones,” which have a spot or a surface that appears black. Comedones also include slightly inflamed, skin colored bumps, as well as “whiteheads,” which have a spot or a surface that appears white. The term “macule” generally refers to a flat spot or area of the skin with a changed color, such as a red spot. The term “pustule” is generally used to refer to an inflamed, pus-filled lesion, or a small inflamed elevation of the skin that is filled with pus. The term “papule” is generally used to refer to a small, solid, usually inflammatory elevation of the skin that does not contain pus. The term “nodule” is generally used to refer to an elevation of a skin that is similar to a papule but is white and dome-shaped. Colloquially, a papule, a pustule or a nodule can be referred to as “a pimple” or “a zit.” The term “cyst” generally refers to an abnormal membranous sac containing a liquid or semi-liquid substance containing white blood cells, dead cells, and bacteria. Cysts can be painful and extend to deeper layers of skin.

In dermatological science and dermatological and cosmetology practice, acne can be classified or categorized into one or more types or categories, according to one or more lines of categorization, such as a predominantly observed type of symptoms, severity of condition or predominant localization. It is to be understood that classification of acne into one of the subtypes does not mean that the characteristics of the classified condition are limited to the symptoms associated with the specific type.

Acne vulgaris is a common form of acne characterized by the appearance of several types of lesions, which may appear together or separately. Individual acne lesions usually last less than two weeks but the deeper papules and nodules may persist for months. Acne vulgaris commonly affects adolescents, but it may also appear, persist or become more severe in adulthood. Acne vulgaris may occur on the face, chest, back and sometimes even more extensively.

Depending on severity, acne can be mild, moderate or severe. Mild acne is generally categorized by the appearance of with blackheads and whiteheads, but can also include papules and pustules, Moderate acne is generally characterized by appearance of more painful, deep-rooted, inflamed lesions, which can result in scarring. Severe acne is characterized by the appearance of deep-rooted inflammatory lesions, including cysts and nodules which can be painful and can produce scarring. Acne conglobata is a category of acne characterized by highly inflammatory cysts that communicate under the skin with abscesses and burrowing sinus tracts.

Some other skin conditions exhibiting acne-like symptoms which can be treated by the compositions and methods described herein are discussed below. Pyoderma faciale, also known as rosacea fulminans, is a condition that appears in females and is characterized by abrupt appearance of inflamed cysts and nodules localized on the face. Rosacea, which can be referred to as acne rosacea, is a condition that can affects both the skin and the eyes and is characterized by redness, bumps, pimples, and, in advanced stages, thickened skin on the nose. In some classification systems, rosacea and acne are considered as separate conditions. Rosacea usually occurs on the face, although the neck and upper chest are also sometimes involved. A mild degree of eye (ocular) involvement occurs in more than fifty percent of people with rosacea. Perioral dermatitis is characterized by the appearance of small tiny papules, pustules, red bumps and scaling with intense itching. It is usually localized to the surrounding area of the mouth and on the chin, or extends to involve the eyelids and the forehead. Gram-negative folliculitis is a bacterial infection characterized by the appearance of pustules and cysts, possibly occurring as a complication resulting from a long term antibiotic treatment of acne vulgaris.

As used herein, the terms “treatment” or “treating” in reference to a skin condition generally mean “having positive effect on a skin condition” and encompass alleviation of at least one symptom of a skin condition, a reduction in the severity of the skin conditions, or delay, prevention, or inhibition of the progression of the skin condition. Treatment need not mean that the condition is totally cured. A composition or a product useful for treatment of a skin condition, or a method of treating a skin condition, needs only to reduce the severity of a skin condition, reduce the severity of symptoms associated therewith, provide improvement to a patient's quality of life, or delay, prevent, or inhibit the onset of symptoms of a skin condition.

### Formulations

In one embodiment, there are provided compositions including dapsone, a first solubilizing agent which is diethylene glycol monoethyl ether, optionally at least one second solubilizing agent, a polymeric viscosity builder, and water, wherein the dapsone is present at a concentration of about 5% w/w to about 10% w/w, about 1% w/w to about 10% w/w, about 3% w/w to about 10% w;/w, about 3% w/w to about 8% w/w, about 4% w/w to about 6% w/w, or about 5%. In certain embodiments, dapsone is present in the composition at 5.0%, 5.5%, 6.0%, 6.5%, 7.0%, 7.5%, 8.0%, 8.5%, 9.0%, 9.5%, or 10.0% w/w.

In some embodiments, the polymeric viscosity builder is an acrylamide/sodium acryloyldimethyltaurate copolymer, and further includes isohexadecane, water, and Polvsorbate 80. In some embodiments, the polymeric viscosity builder is present at a concentration of about 2% w/w to about 6% w/w. In some embodiments, the polymeric viscosity builder is present at a concentration of about 3% w/w to about 5% w/w. In some embodiments, the polymeric viscosity builder is present in the composition at about 4% wAv. An example of a commercially available polymeric viscosity builder including acrylamide/sodium acryloyldimethyltaurate copolymer is Sepineo P 600, the MSDS of which is incorporated by reference in its entirety.

In some embodiments, diethylene glycol monoethyl ether is present at a concentration of about 25% w/w to about 40% w/w. In some embodiments, diethylene glycol monoethyl ether is present at a concentration of about 30% w/w to about 40% w/w. In some embodiments, diethylene glycol monoethyl ether is present at a concentration of about 35% w/w to about 40% w/w.

In some embodiments, diethylene glycol monoethyl ether is present at a concentration of about 10% w/w to about 40% W/W, about 20% w/w to about 30% w/w, or about 25% w/w. In some embodiments, diethylene glycol monoethyl ether is present at a concentration of about 30% w/w.

In some embodiments, the second solubilizing agent is selected from alcohols, glycols, esters, ethers, or silicones. Such second solubilizing agents include, but are not limited to, PEG 400, lactic acid, dimethyl isosorbide, propylene glycol, propylene carbonate, hexylene glycol, isostearyl alcohol, benzyl alcohol, diethyl sebacate, and ethanol.

In certain embodiments, the second solubilizing agent is propylene glycol. In some embodiments, propylene glycol is present at a concentration of about 2% w/w to 8% w/w. In some embodiments, propylene glycol is present at a concentration of about 3% w/w to 7% w/w. In some embodiments, propylene glycol is present in the composition at about 5% w/w,

In certain embodiments, the second solubilizing agent is propylene carbonate. In some embodiments, propylene carbonate is present at a concentration of about 2% w/w to 8% w/w. In some embodiments, propylene carbonate is present at a concentration of about 3% w/w to 7% w/w. In some embodiments, propylene carbonate is present in the composition at about 5% w/w.

In certain embodiments, the second solubilizing agent is ethanol. In some embodiments, ethanol is present at a concentration orf about 1% w/w to about 5% w/w. In some embodiments, ethanol is present at a concentration of about 2% w/w to about 4% w/w. In some embodiments, ethanol is present in the composition at about 3% w/w.

In some embodiments, the compositions further include methyl paraben.

In other embodiments, the compositions further include carbomer homopolymer type C. In some embodiments, carbomer homopolymer type C is present at a concentration of about 0.7% w/w to about 1.5% w/w. In other embodiments, carbomer homopolymer type C is present at a concentration of about 0.85% w/w to about 1.0% w/w.

In some embodiments, the compositions further include a neutralizing agent. In certain embodiments, the neutralizing agent is an ionic or amine buffer. In certain embodiments, the neutralizing agent is sodium hydroxide or triethanolamine. Use of a neutralizing agent results in compositions typically having a from 5.5 to 6.5.

In some embodiments, the compositions further include a chelating agent. In some embodiments, the chelating agent is ethylene diamine tetraacetic acid (EDTA). EDTA is typically present in the compositions from about 0.02% w/w to about 0.04% w/w. In certain embodiments, EDTA is present in the compositions at about 0.03% w/w.

Compositions described herein are typically in the form of a gel, an emulsion, a cream, a liquid, a paste, a lotion, a nanoemulsion, a microemuision, a reverse emulsion, or a liposomal cream. For example, in an embodiment, the composition can be a 7.5% w/w dapsone gel contained in a pump dispenser.

### Methods of Treatment

According to some embodiments, methods of treating acne vulgaris are provided using the dapsone formulations described herein.

In an embodiment, a patient having acne vulgaris can perform a treatment regimen for a period of time effective to improve the acne vulgaris. The regimen can include applying a dapsone gel formulation as described herein at a frequency of once a day to the face of the patient. In some embodiments the regimen can include applying a 7.5% w/w dapsone gel formulation as described herein at a frequency of once a day to the face of the patient. In some embodiments the regimen can include applying a gel formulation comprising about 7.5% w/w dapsone, about 40% w/w diethylene glycol monoethyl ether, and about 4% acrylamide/sodium acryloyldimethyltaurate copolymer based thickener as described herein at a frequency of once a day to the face of the patient.

According to some embodiments, the treatment regimen can be performed at a sufficient frequency for a period of time effective to improve the acne vulgaris. In some embodiments, the treatment regimen can be performed only once daily. When the treatment regimen is performed once daily, it can be performed at various times such as at night or in the morning.

In some embodiments, the treatment regimen can be performed for a treatment duration effective to improve the acne vulgaris. In some embodiments, the treatment duration effective to improve the acne vulgaris can be about 12 weeks. The treatment duration effective to improve the acne vulgaris can be about 4 weeks, about 8 weeks, about 10 weeks, and the like. According to some embodiments, the treatment duration effective to improve the acne vulgaris can be about 12 weeks or more, about 10 weeks or more, about 8 weeks or more, about 4 weeks or more, and the like. In some embodiments, the treatment duration effective to improve the acne vulgaris can be in the range of about 2 weeks to about 12 weeks. In some embodiments, the treatment duration effective to improve the acne vulgaris can be in the range of about 4 weeks to about 12 weeks. In some embodiments, the treatment duration effective to improve the acne vulgaris can be in the range of about 8 weeks to about 12 weeks. According to some embodiments, the treatment duration effective to improve the acne vulgaris can be determined by a patient's physician.

In some embodiments, an improvement in acne vulgaris can include a reduction in the severity of a patient's acne vulgaris. For example, an improvement in acne vulgaris can, for example, include a reduction in the number of inflammatory and/or non-inflammatory lesions, comedones, papules/pustules or nodulocystic lesions present on the face of the patient with acne vulgaris. In some embodiments, improvement can be present where a patient's nodules change from inflammatory to non-inflammatory. According to some embodiments, an improvement in acne vulgaris can include a reduction of the severity of the acne vulgaris to clear (e.g., no or nearly no evidence of acne vulgaris) or almost clear (e.g. rare non-inflammatory lesions present, with rare non-inflamed papules) as assessed by a physician and/or self-assessed by the patient.

A topical gel formulation can be provided as described above. For example, in an embodiment, a topical gel formulation can be provided comprising dapsone, the dapsone being present in the topical gel formulation in an amount of about 7.5% by weight, based on the total weight of the topical gel formulation.

In some embodiments, the topical dapsone gel formulation can be applied to the face of a patient having acne vulgaris. According to some embodiments, the topical dapsone gel formulation can be applied to the entire face of the patient. After the topical dapsone gel formulation is applied to the patient's face, the topical dapsone gel formulation can be rubbed into the entire face of the patient. In some embodiments, the topical dapsone gel formulation can be rubbed into the entire face of the patient except for the eyes, mouth, and areas immediately surrounding the eyes and/or mouth. In some embodiments, the topical dapsone gel formulation may only be applied to those areas of the face exhibiting the symptoms of acne.

According to some embodiments, the topical dapsone gel formulation can be left on the face for an extended period of time after it is applied. In such embodiments, a patient should not bathe or shower during that extended period of time. In some embodiments, the extended period of time can be about 8 hours or more, about 6 hours or more, about 4 hours or more, and the like.

### Post Inflammatory Hyperpigmentation

According to some embodiments, methods of treating post-inflammatory hyperpigmentation are provided using the dapsone formulations described herein. Post-inflammatory hyperpigmentation (PIH) is a condition in which an injury or inflammation to the skin causes increased pigment production. PIH occurs in darker-skinned individuals (e.g. Fitzpatrick type 5 or 6) and can be difficult to treat. The most common cause of PIH is acne. but it also can result from psoriasis, a burn, or an injury. In some embodiments, the PIH treated is caused by acne vulgaris.

In an embodiment, a patient having PIH can perform a treatment regimen for a period of time effective to improve the PIH. The regimen can include applying a dapsone gel formulation as described herein at a frequency of once a day to the face of the patient. In some embodiments the regimen can include applying a 7.5% w/w dapsone gel formulation as described herein at a frequency of once a day to the face of the patient. In some embodiments the regimen can include applying a gel formulation comprising about 7.5% w/w dapsone, about 40% w/w diethylene glycol monoethyl ether, and about 4% of an acrylamide/sodium acryloyldimethyltaurate copolymer based thickener as described herein at a frequency of once a day to the face of the patient.

According to some embodiments, the treatment regimen can be performed at a sufficient frequency for a period of time effective to improve a patient's post inflammatory hyperpigmentation. In some embodiments, the treatment regimen can be performed only once daily. When the treatment regimen is performed once daily, it can be performed at various times such as at night or in the morning.

In some embodiments, the treatment regimen can be performed for a treatment duration effective to improve the PIH. In some embodiments, the treatment duration effective to improve the PIH can be about 12 weeks. The treatment duration effective to improve the PIH can be about 4 weeks, about 8 weeks, about 10 weeks, and the like. According to some embodiments, the treatment duration effective to improve the PIH can be about 12 weeks or more, about 10 weeks or more, about 8 weeks or more, about 4 weeks or more, and the like. In some embodiments, the treatment duration effective to improve the PIH can be in the range of about 2 weeks to about 12 weeks. In some embodiments, the treatment duration effective to improve the PIH can be in the range of about 4 weeks to about 12 weeks. In some embodiments, the treatment duration effective to improve the PIH can be in the range of about 8 weeks to about 12 weeks. According to some embodiments, the treatment duration effective to improve the PIH can be determined by a patient's physician.

In some embodiments, an improvement in PIH can include a reduction in the severity of a patient's PIH. For example, an improvement in PIH can, for example, include a reduction in the number of dark spots and/or a lightening of the dark spots present on the face of the patient with PIH. In some embodiments, an improvement in PIH can include the total clearing of dark spots on the face of the patient.

In some embodiments, the methods of treatment can be effective to treat a patient having both acne vulgaris and PIH. For example, a patient with acne vulgaris could use the formulations and methods described above to treat their acne, then treat the PIH resulting from the acne vulgaris with the same formulations and methods.

### EMBODIMENTS

The following example dapsone formulation embodiments are specifically contemplated herein.

Embodiment 1. A composition comprising dapsone, a first solubilizing agent which is diethylene glycol monoethyl ether, optionally at least one second solubilizing agent, a polymeric viscosity builder, and water, wherein the dapsone is present in the composition at a concentration of about 3% w/w to about 10% w/w.

Embodiment 2. The composition of embodiment 1, wherein the diethylene glycol monoethyl ether is present at a concentration of about 10% w/w to about 40% w/w.

Embodiment 3. The composition of embodiment 1, wherein the diethylene glycol monoethyl ether is present at a concentration of about 20% w/w to about 30% w/w.

Embodiment 4. The composition of embodiment 1, wherein the diethylene glycol monoethyl ether is present in the composition at a concentration of about 25% w/w.

Embodiment 5. The composition of embodiment 1 wherein the second solubilizing agent is selected an alcohol, a glycol, an ester, or an ether.

Embodiment 6. The composition of embodiment 1, wherein the second solubilizing agent is PEG 400, lactic acid, dimethyl isosorbide, propylene glycol, propylene carbonate, hexylene glycol, isostearyl alcohol, diethyl sebacate, or ethanol.

Embodiment 7. The composition of embodiment 6, wherein the second solubilizing agent is propylene glycol.

Embodiment 8. The composition of embodiment 7, wherein the propylene glycol is present in the composition at a concentration of about 5% w/w.

Embodiment 9. The composition of embodiment 6, wherein the second solubilizing agent is propylene carbonate.

Embodiment 10. The composition of embodiment 9, wherein the propylene carbonate is present in the composition at a concentration of about 5% w/w.

Embodiment 11. The composition of embodiment 6, wherein the second solubilizing agent is ethanol.

Embodiment 12. The composition of embodiment 11, wherein the ethanol is present in the composition at a concentration of about 3% w/w.

Embodiment 13. The composition of embodiment 1, wherein the polymeric viscosity builder comprises an acrylamide/sodium acryloyldimethyltaurate copolymer.

Embodiment 14. The composition of embodiment 1, wherein the polymeric viscosity builder is present at a concentration of about 2% w/w to about 6% w/w.

Embodiment 15. The composition of embodiment 1, wherein the polymeric viscosity builder is present at a concentration of about 4% w/w.

Embodiment 16. The composition of embodiment 1, further comprising methyl paraben.

Embodiment 17. The composition of embodiment 1, further comprising Carbomer interpolymer type A, Carbomer interpolymer type B, or Carbomer Homopolymer Type C.

Embodiment 18. The composition of embodiment 17, wherein the Carbomer Homopolymer Type C is present at a concentration of about 0.7% w/w to about 1.5% w/w.

Embodiment 19. The composition of embodiment 17, wherein the Carbomer Homopolymer Type C is present at a concentration of about 0.85% w/w to about 1.5% w/w.

Embodiment 20. The composition of embodiment 17, wherein the Carbomer interpolymer Type A is present at a concentration of about 1% w/w to 2% w/w.

Embodiment 21. The composition of embodiment 17, wherein the Carbomer interpolymer Type B is present at a concentration of about 0.1% w/w to about 0.5% w/w.

Embodiment 22. The composition of embodiment 1, further comprising a neutralizing agent.

Embodiment 23. The composition of embodiment 22 wherein the neutralizing agent is NaOH or triethanolamine.

Embodiment 24. The composition of embodiment 1 further comprising a chelating agent.

Embodiment 25. The composition of embodiment 24, wherein the chelating agent is ethylene diamine tetraacetic acid.

Embodiment 26. The composition of embodiment 25, wherein the ethylene diamine tetraacetic acid is present at a concentration of about 0.02% w/w to about 0.04% w/w.

Embodiment 27. The composition of embodiment 25, wherein the ethylene diamine tetraacetic acid is present in the composition at about 0.03% w/w.

Embodiment 28. The composition of embodiment 1 wherein the composition is in the form of a gel, a suspension, an emulsion, a cream, a liquid, a paste, a lotion, a nanoemulsion, a microemulsion, a reverse emulsion, or a liposomal cream.

Embodiment 29. A method for treating a dermatological condition comprising administering to a subject in need thereof a therapeutically effective amount of a composition of embodiment 1.

Embodiment 30. The method of embodiment 29 wherein the condition is acne vulgaris, rosacea, atopic dermatitis, treatment of chronic wounds, bed sores, keratosis piralis, sebaceous cysts, inflammatory dermatoses, post inflammatory hyperpigmentation, eczema, xerosis, pruritis, lichen planus, nodular prurigo, dermatitis, eczema, or miliaria.

Embodiment 31. The method of embodiment 30 wherein the condition is acne vulgaris.

Embodiment 32. The composition of embodiment 1, 2, 3, 4, 30, or 31, wherein the second solubilizing agent is selected an alcohol, a glycol, an ester, or an ether.

Embodiment 33. The composition of embodiment 1, 2, 3, 4, 30, 31, or 32, wherein the second solubilizing agent is PEG 400, lactic acid, dimethyl isosorbide, propylene glycol, propylene carbonate, hexylene glycol, isostearyl alcohol, diethyl sebacate, or ethanol.

Embodiment 34. The composition of embodiment 33, wherein the second solubilizing agent is propylene glycol.

Embodiment 35. The composition of embodiment 34, wherein the propylene glycol is present in the composition at a concentration of about 5% w/w.

Embodiment 36. The composition of embodiment 33, wherein the second solubilizing agent is propylene carbonate.

Embodiment 37. The composition of embodiment 36, wherein the propylene carbonate is present in the composition at a concentration of about 5% w/w.

Embodiment 38. The composition of embodiment 33, wherein the second solubilizing agent is ethanol.

Embodiment 39. The composition of embodiment 38, wherein the ethanol is present in the composition at a concentration of about 3% w/w.

Embodiment 40. The composition of embodiment 1, 2, 3, 4, 30, 31, 32, 33, 34, 35, 36, 37, 38, or 39, wherein the polymeric viscosity builder comprises an acrylamide/sodium acryloyldimethyltaurate copolymer.

Embodiment 41. The composition of embodiment 1, 2, 3, 4, 30, 31, 32, 33, 34, 35, 36, 37, 38, 39, or 40 wherein the polymeric viscosity builder is present at a concentration of about 2% w/w to about 6% w/w.

Embodiment 42. The composition of embodiment 41, wherein the polymeric viscosity builder is present at a concentration of about 4% w/w.

Embodiment 43. The composition of embodiment 1, 2, 3, 4, 30, 31, 32, 33, 34, 35, 36, 37, 38, 39, 40, 41, or 42, further comprising methyl paraben.

Embodiment 44. The composition of embodiment 1, 2, 3, 4, 30, 31, 32, 33, 34, 35, 36, 37, 38, 39, 40, 41, 42, or 43 further comprising Carbomer interpolymer type A, Carbomer interpolymer type B, or Carbomer Homopolymer Type C.

Embodiment 45. The composition of embodiment 44, wherein the Carbomer Homopolymer Type C is present at a concentration of about 0.7% w/w to about 1.5% w/w.

Embodiment 46. The composition of embodiment 44, wherein the Carborner Homopolymer Type C is present at a concentration of about 0.85% w/w to about 1.5% w/w.

Embodiment 47. The composition of embodiment 44, wherein the Carbomer interpolymer Type A is present at a concentration of about 1% w/w to 2% w/w.

Embodiment 48. The composition of embodiment 44, wherein the Carbomer interpolymer Type B is present at a concentration of about 0.1% w/w to about 0.5% w/w.

Embodiment 49. The composition of embodiment 1, 3, 4, 30, 31, 32, 33, 34, 35, 36, 37, 38, 39, 40, 41, 42, 43, 44, 45, 46, 47, or 48 further comprising a neutralizing agent.

Embodiment 50. The composition of embodiment 49 wherein the neutralizing agent is NaOH or triethanolamine.

Embodiment 51. The composition of embodiment 1, 2, 3, 4, 30, 31, 32, 33, 34, 35, 36, 37, 38, 39, 40, 41, 42, 43, 44, 45, 46, 47, 48, 49, or 50 further comprising a chelating agent.

Embodiment 52. The composition of embodiment 51, wherein the chelating agent is ethylene diamine tetraacetic acid.

Embodiment 53. The composition of embodiment 52, wherein the ethylene diamine tetraacetic acid is present at a concentration of about 0.02% w/w to about 0.04% w/w.

Embodiment 54. The composition of embodiment 52, wherein the ethylene diamine tetraacetic acid is present in the composition at about 0.03% w/w.

Embodiment 55. The composition of embodiment 1, 2, 3, 4, 30, 31, 32, 33, 34, 35, 36, 37, 38, 39, 40, 41, 42, 43, 44, 45, 46, 47, 48, 49, 50, 51, 52, 53, or 54 wherein the composition is in the form of a gel, a suspension, an emulsion, a cream, a liquid, a paste, a lotion, a nanoemulsion, a microemulsion, a reverse emulsion, or a liposomal cream.

Embodiment 56. A method for treating a dermatological condition comprising administering to a subject in need thereof a therapeutically effective amount of a composition of embodiment 1, 2, 3, 4, 30, 31, 32, 33, 34, 35, 36, 37, 38, 39, 40, 41, 42, 43, 44, 45, 46, 47, 48, 49, 50, 51, 52, 53, 54 or 55.

Embodiment 57. The method of embodiment 56 wherein the condition is acne vulgaris, rosacea, atopic dermatitis, treatment of chronic wounds, bed sores, keratosis piralis, sebaceous cysts, infiamrnatory dermatoses, post inflammatory hyperpigmentation, eczema, xerosis, pruritis, lichen planus, nodular prurigo, dermatitis, eczema, or miliaria.

Embodiment 58. The method of embodiment 56 wherein the condition is acne vulgaris.

The following examples are intended only to illustrate the some embodiments and should in no way be construed as limiting the claims.

### EXAMPLES

### Example 1

Table 1 lists two formulations containing equivalent levels of diethylene glycol monoethyl ether) that show the impact of acrylamide/sodium acryloyidimethyltaurate copolymer based thickener on dapsone particle size.

TABLE 1
Formulations Tested For Dapsone Crystal Size

| | Formulation # | |
|---|---|---|
| | ENA | ENC |
| Dapsone | 7.5 | 7.5 |
| Diethylene glycol monoethyl ether | 30 | 30 |
| Carbomer homopolymer type C. | — | 1 |
| acrylamide/sodium | 4 | — |
| acryloyldimethyltaurate copolymer | | |
| based thickener | | |
| Methyl paraben | 0.2 | 0.2 |
| pH adjusting solution | pH 5.5-7 | pH 5.5-7 |
| Purified Water | Q.S 100 | Q.S 100 |

### Example 2

Anti-oxidants and chelating agents such as sodium metabisulfite, citric acid and EDTA were added to formulations to help slow down or completely stop any impurity formation. Table 2 presents the composition of formulations tested. Formulation A7 with sodium metabisulfite minimized the intensity of yellow color caused by the increased solubility of dapsone in diethylene glycol monoethyl ether and maintained the low color intensity over time at accelerated condition (400 C).

TABLE 2
Compositions Tested containing Antioxidants or Chelating Agents

| | Composition # | | |
|---|---|---|---|
| | A5 | A6 | 47 |
| Dapsone | 7.5 | | |
| Diethylene glycol monoethyl | 35 | 40 | 35 |
| ether | | | |
| carbomer homopolyme type C | 1.25 | — | 1.25 |
| Acrylamide/sodium | — | 4 | — |
| acryloyldimethyltaurate | | | |
| copolymer emulsion | | | |

| | | |
|---|---|---|
| EDTA | 0.05 | — |
| Anhydrous Citric Acid | 0.1 | — |
| Sodium Metabisullite | — | 0.2 |
| Methyl paraben | 0.17 | 0.2 |
| Propyl paraben | 0.03 | — |

| | |
|---|---|
| NaOH/pH adjusting solution | pH 5.5-6.5 |
| Purified Water | Q.S 100 |

### Example 3

Additional example compositions contemplated for use as described herein are set forth in Table 3 below.

TABLE 3
Additional examples containing alternate neutralizer

| | % w/w | | | | | |
|---|---|---|---|---|---|---|
| Materials | 5-1 | 5-2 | 5-3 | 5-4 | 5-5 | 5-6 |
| Dapsone | 7.5 | | | | | |
| Diethylene glycol monoethyl ether | 30 | 35 | 40 | 30 | 40 | 25 |

| | |
|---|---|
| carbomer homopolymer type C | 1 |
| Methylparaben | 0.2 |
| Triethanolamine (TEA) Q.S. | pH 5.5-6.5 |
| Hydrochloric Acid Q.S | pH 5.5-6.5 |
| Purified Water | q.s.a.d.100 |

### Example 4

Additional example compositions contemplated for use as described herein are set forth in Table 4 below.

TABLE 4
Additional examples (containing co-solvents,
stabilizer and alternate thickener)

| | % w/w | | | | | |
|---|---|---|---|---|---|---|
| Materials | 6-1 | 6-2 | 6-3 | 6-4 | 6-5 | 6-6 |
| Dapsone | 7.5 | | 10 | 7.5 | | |
| Diethylene glycol monoethyl ether | 25 | 35 | 35 | 25 | 30 | 40 |

| Propylene glycol | 5 |
|---|---|
| Propylene Carbonate | 5 |

| | | | |
|---|---|---|---|
| Ethanol (absolute) | 3 | — | 3 |

| EDTA | 0.03 | | |
|---|---|---|---|
| Carboraer Interpolymer Type A | — | 1.5 | |
| Carbomer Interpolymer Type B | — | 0.3 | |
| Acrylamide/sodium | 4 | — | 4 |
| acryloyldimethyltaurate | | | |
| Methyl Paraben | 0.2 | | |
| Triethanolamine | — | Q.S. pH 5.5-6.5 | |

| | |
|---|---|
| Purified Water | q.s.a.d.100 |

### Example 5

### Clinical Studies

Two Phase 3 clinical studies investigated the safety and efficacy of the use of a 7.5% w/w dapsone gel compared to vehicle for the treatment of acne vulgaris.

The formulations used in the studies included the formulation elements listed below:

| | Dapsone gel | Vehicle |
|---|---|---|
| Formulation | % (w/w) | % (w/w) |
| Dapsone | 7.5 | — |
| Diethylene glycol monoethyl ether | 30 | 30 |
| acrylamide/sodium | 4 | 4 |
| acryloyldimethyltaurate copolymer | | |
| based thickener | | |
| Methyl paraben | 0.2 | 0.2 |
| pH adjusting solution | pH 5.5-7 | pH 5.5-7 |
| Purified Water | Q.S 100 | Q.S 100 |

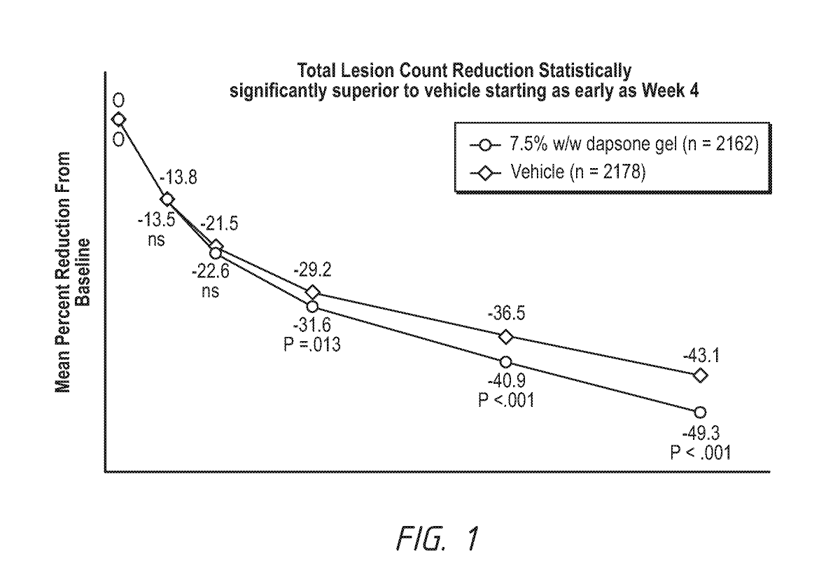

A total of 4340 patients were enrolled in the Phase 3 studies. The patients enrolled in the studies were 12 years and older with 20-50 inflammatory lesions and about 30-100 noninflammatory lesions. Patients were randomized in a 1:1 ratio by study coordinators to one of two treatment groups. In the first group, patients administered the topical dapsone gel formulation containing 7.5% w/w dapsone to their entire face once a day. In the second group, patients administered the vehicle formulation once a day. Patients were treated for twelve weeks. The patients were assessed by a physician throughout the trial for reduction in inflammatory and non-inflammatory lesions and adverse events at weeks 0, 1, 2, 4, 8 and 12. The overall reduction in lesions in the pooled results of the two studies is illustrated in FIG. 1.

Surprisingly, the mean percent reduction in total lesions was statistically significantly superior to vehicle, starting at week 4 and continuing through to week 12. Both inflammatory and non inflammatory were reduced significantly. Specifically, the mean percentage of total lesion reduction in patients administering the topical dapsone gel formulation containing 7.5% w/w dapsone to their entire face once a day was −31.6% at week 4, −40.9% at week 8, and −49.3% at week 12. At week 12, inflammatory lesions were reduced by 15.8 lesions (54.6%; n=2162) vs 13.9 lesions with vehicle (48.1%; n=2178), and non-inflammatory lesions were reduced by 20.7 lesions (45.1%) vs 18.0 lesions with vehicle (39.4%). The Global Acne Assessment Score “GAAS” success rate in patients was 29.8% (n=2162) vs 21.1% with vehicle (n=2178).

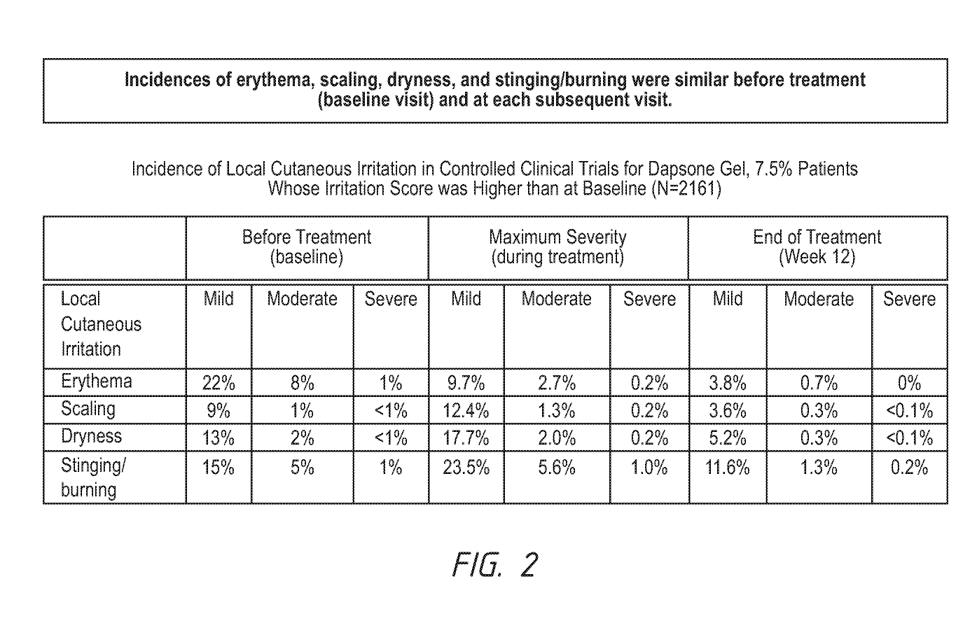

The patients were also assessed for erythema, scaling, dryness, and stinging/burning throughout the trials. A chart showing the incidence of local cutaneous irritation in patients whose irritation score was high than at baseline is shown in FIG. 2. Surprisingly, the amount and severity of erythema, scaling, dryness, and stinging/burning was reduced over the treatment period (between baseline and end of treatment at 12 weeks). Also, during the study, the formulation was extremely well tolerated. Less than 1% of total patients experienced redness, dryness and peeling of the treated skin.

While improvements in acne severity were significant for all subgroups of age, gender and race, improvements were even greater in adults compared to adolescents and in females compared to males.

During the same trials, it was also discovered that a statistically significant resolution of postinflammatory hyperpigmentation in patients. Specifically, patients with darker skin (Fitzpatrick type 5 or 6) experienced accelerated resolution of postinflammatory hyperpigmentation caused by acne lesions. A reduction in the number of dark spots was observed, and a significant number of patients reported no dark spots at week 12 of treatment.

While this some embodiments have been described with respect to these specific examples, it is understood that other modifications and variations are possible without departing from the spirit of the invention. Each and every reference identified herein is incorporated by reference in its entirety.

Attached herewith is the prescribing information for ACZONE® Gel, 7.5%, which is an embodiment of the formulations and methods of treatment described herein.

### Full Prescribing Information

1 Indications and Usage

ACZONE® (dapsone) Gel, 7.5%, is indicated for the topical treatment of acne vulgaris in patients 12 years of age and older.

2 Dosage and Administration

For topical use only. Not for oral, ophthalmic, or intravaginal use.

After the skin is gently washed and patted dry, apply approximately a pea-sized amount of ACZONE Gel, 7.5%, in a thin layer to the entire face once daily. In addition, a thin layer may be applied to other affected areas once daily. Rub in ACZONE Gel, 7.5%, gently and completely.

If there is no improvement after 12 weeks, treatment with ACZONE Gel, 7.5% should be reassessed (2).

3 Dosage Forms and Strengths

Gel, 7.5%. Each gram of ACZONE Gel, 7.5% contains 75 mg of dapsone in an off-white to yellow gel with suspended particles.

4 Contraindications

None.

5 Warnings and Precautions

5.1 Hematological Effects

Methemoglobinemia

Cases of methemoglobinemia, with resultant hospitalization, have been reported postmarketing in association with twice daily dapsone gel, 5%, treatment. Patients with glucose-6-phosphate dehydrogenase deficiency or congenital or idiopathic methemoglobinemia are more susceptible to drug-induced methemoglobinemia. Avoid use of ACZONE Gel. 7.5% in those patients with congenital or idiopathic methemoglobinemia.

Signs and symptoms of methemoglobinemia may be delayed some hours after exposure. Initial signs and symptoms of methemoglobinemia are characterized by a slate grey cyanosis seen in e.g., buccal mucous membranes, lips, and nail beds. Advise patients to discontinue ACZONE Gel, 7.5% and seek immediate medical attention in the event of cyanosis.

Dapsone can cause elevated methemoglobin levels particularly in conjunction with methemoglobin-inducing agents [see Drug Interactions (7.4)].

Hemolysis

Oral dapsone treatment has produced dose-related hemolysis and hemolytic anemia. Individuals with glucose-6-phosphate dehydrogenase (G6PD) deficiency are more prone to hemolysis with the use of certain drugs. G6PD deficiency is most prevalent in populations of African, South Asian, Middle Eastern, and Mediterranean ancestry.

In clinical trials, there was no evidence of clinically relevant hemolysis or hemolytic anemia in subjects treated with topical dapsone. Some subjects with G6PD deficiency using dapsone gel, 5%, twice daily developed laboratory changes suggestive of hemolysis [see Use in Specific Populations (8.6)].

Discontinue ACZONE Gel, 7.5%, if signs and symptoms suggestive of hemolytic anemia occur. Avoid use of ACZONE Gel, 7.5% in patients who are taking oral dapsone or antimalarial medications because of the potential for hemolytic reactions. Combination of ACZONE Gel, 7.5%, with trimethoprim/sulfamethoxazole (TMP/SMX) may increase the likelihood of hemolysis in patients with G6PD deficiency [see Drug Interactions (7.1)].

5.2 Peripheral Neuropathy

Peripheral neuropathy (motor loss and muscle weakness) has been reported with oral dapsone treatment. No events of peripheral neuropathy were observed in clinical trials with topical dapsone treatment.

5.3 Skin Reactions

Skin reactions (toxic epidermal necrolysis, erythema multiforme, morbilliform and scarlatiniform reactions, bullous and exfoliative dermatitis, erythema nodosum, and urticaria) have been reported with oral dapsone treatment. These types of skin reactions were not observed in clinical trials with topical dapsone treatment.

6 Adverse Reactions

6.1 Clinical Studies Experience

Because clinical trials are conducted under widely varying conditions, adverse reaction rates observed in the clinical trials of a drug cannot be directly compared to rates in the clinical trials of another drug and may not reflect the rates observed in practice.

A total of 2161 subjects were treated with ACZONE Gel, 7.5%, for 12 weeks in 2 controlled clinical trials. The population ranged in age from 12 to 63 years, was 56% female, and 58% Caucasian. Adverse drug reactions that were reported in at least 0.9% of subjects treated with ACZONE Gel, 7.5% appear in Table 1 below.

TABLE 1
Adverse Reactions Occurring in at Least 0.9% of Subjects with
Acne Vulgaris in 12-week Controlled Clinical Trials

| | ACZONE Gel, 7.5% | Vehicle |
|---|---|---|
| | (N = 2161) | (N = 2175) |
| Application Site Dryness | 24 (1.1%) | 21 (1.0%) |
| Application Site Pruritus | 20 (0.9%) | 11 (0.5%) |

6.2 Experience with Oral Use of Dapsone

Although not observed in the clinical trials with topical dapsone, serious adverse reactions have been reported with oral use of dapsone, including agranulocytosis, hemolytic anemia, peripheral neuropathy (motor loss and muscle weakness), and skin reactions (toxic epidermal necrolysis, erythema multiforme, morbilliform and scarlatiniform reactions, bullous and exfoliative dermatitis, erythema nodosum, and urticaria).

6.3 Postmarketing Experience

The following adverse reactions have been identified during post-approval use of topical dapsone. Because these reactions are reported voluntarily from a population of uncertain size, it is not always possible to reliably estimate their frequency or establish a causal relationship to drug exposure.

Methemoglobinemia has been identified during postmarketing use of topical dapsone [see Warnings and Precautions (5.1)].

7 Drug Interactions

No formal drug-drug interaction studies were conducted with ACZONE Gel, 7.5%.

7.1 Trimethoprim-Sulfamethoxazole

A drug-drug interaction study evaluated the effect of the use of dapsone gel, 5% in combination with double strength (160 mg/800 mg) trimethoprim-sulfamethoxazole (TMP/SMX). During co-administration, systemic levels of TMP and SMX were essentially unchanged, however, levels of dapsone and its metabolites increased in the presence of TMP/SMX. The systemic exposure from ACZONE Gel, 7.5% is expected to be about 1% of that from the 100 mg oral dose, even when co-administered with TMP/SMX.

7.2 Topical Benzoyl Peroxide

Topical application of dapsone gel followed by benzoyl peroxide in patients with acne vulgaris may result in a temporary local yellow or orange discoloration of the skin and facial hair.

7.3 Drug Interactions with Oral Dapsone

Certain concomitant medications (such as rifampin, anticonvulsants, St. John's wort) may increase the formation of dapsone hydroxylamine, a metabolite of dapsone associated with hemolysis. With oral dapsone treatment, folic acid antagonists such as pyrimethamine have been noted to possibly increase the likelihood of hematologic reactions.

7.4 Concomitant Use with Drugs that Induce Methemoglobinemia

Concomitant use of ACZONE Gel, 7.5% with drugs that induce methemoglobinemia such as sulfonamides, acetaminophen, acetanilide, aniline dyes, benzocaine, chloroquine, dapsone, naphthalene, nitrates and nitrites, nitrofurantoin, nitroglycerin, nitroprusside, pamaquine, para-aminosalicylic acid, phenacetin, phenobarbital, phenytoin, primaquine, and quinine may increase the risk for developing methemoglobinemia [see Warnings and Precautions (5.1)].

8 Use in Specific Populations

8.1 Pregnancy

Teratogenic Effects: Pregnancy Category C

There are no adequate and well controlled studies in pregnant women. ACZONE Gel. 7.5%, should be used during pregnancy only if the potential benefit justifies the potential risk to the fetus. Dapsone has been shown to have an embryocidal effect in rats and rabbits when administered orally during the period of organogenesis in doses of 75 mg/kg/day and 150 mg/kg/day, respectively (approximately 1400 and 425 times, respectively, the systemic exposure that is associated with the maximum recommended human dose (MRHD) of ACZONE Gel, 7.5%, based on AUC comparisons). These effects may have been secondary to maternal toxicity.

8.3 Nursing Mothers

Although systemic absorption of dapsone following topical application of ACZONE Gel, 7.5%, is minimal relative to oral dapsone administration, it is known that dapsone is excreted in human milk. Because of the potential for oral dapsone to cause adverse reactions in nursing infants, a decision should be made whether to discontinue nursing or to discontinue ACZONE Gel. 7.5%, taking into account the importance of the drug to the mother.

8.4 Pediatric Use

Safety and efficacy was evaluated in 1066 subjects aged 12-17 years old treated with ACZONE Gel, 7.5% in the clinical trials. The safety profile for ACZONE Gel, 7.5%, was similar to the vehicle control group. Safety and effectiveness of ACZONE Gel, 7.5%, have not been established in pediatric patients below the age of 12 years.

8.5 Geriatric Use

Clinical trials of ACZONE Gel, 7.5% did not include sufficient numbers of subjects aged 65 years and over to determine whether they respond differently from younger subjects.

8.6 Glucose-6-phosphate Dehydrogenase (G6PD) Deficiency

Individuals with glucose-6-phosphate dehydrogenase (G6PD) deficiency may be more prone to methemoglobinemia and hemolysis [see Warnings and Precautions (5.1)].

ACZONE Gel, 5% and vehicle were evaluated in a randomized, double-blind, cross-over design clinical study of 64 subjects with G6PD deficiency and acne vulgaris. Subjects were Black (88%), Asian (6%), Hispanic (2%) or of other racial origin (5%). Blood samples were taken at Baseline, Week 2, and Week 12 during both vehicle and ACZONE Gel, 5% treatment periods. Some of these subjects developed laboratory changes suggestive of hemolysis, but there was no evidence of clinically significant hemolytic anemia in this study [see Warnings and Precautions (5.1)].

11 Description

ACZONE (dapsone) Gel, 7.5%, contains dapsone, a sulfone, in an aqueous gel base for topical dermatologic use. ACZONE Gel, 7.5% is an off-white to yellow gel with suspended particles. Chemically, dapsone has an empirical formula of C₁₂H₁₂N₂O₂S. It is a white or slightly yellow-white, crystalline powder that has a molecular weight of 248.30. Dapsone's chemical name is 4-[(4-aminobenzene) sulfonyl] aniline and its structural formula is:

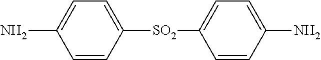

Each gram of ACZONE Gel, 7.5%, contains 75 mg of dapsone, USP, in a gel of diethylene glycol monoethyl ether, methylparaben, acrylamide/sodium acryloyldimethyl taurate copolymer, isohexadecane, polysorbate 80, and purified water.

12 Clinical Pharmacology

12.1 Mechanism of Action

The mechanism of action of dapsone gel in treating acne vulgaris is not known.

12.3 Pharmacokinetics

In a pharmacokinetic study, male and female subjects 16 years of age or older with acne vulgaris (N=19) received 2 grains of ACZONE Gel, 7.5%, topically to the face, upper chest, upper back and shoulders once daily for 28 days. Steady state for dapsone was reached within 7 days of dosing. On Day 28, the mean dapsone maximum plasma concentration (Cmax) and area under the concentration-time curve from 0 to 24 hours post dose (AUC₀₋₂₄ₕ) were 13.0±6.8 ng/mL and 282±146 ng·h/mL, respectively. The systemic exposure from ACZONE Gel, 7.5% is expected to be about 1% of that from a 100 mg oral dose.

Long-term safety studies were not conducted with ACZONE Gel, 7.5%, however, in a long-term clinical study of dapsone gel, 5% treatment (twice daily), periodic blood samples were collected up to 12 months to determine systemic exposure of dapsone and its metabolites in approximately 500 subjects. Based on the measurable dapsone concentrations from 408 subjects (M=192, F=216), obtained at Month 3, neither gender nor race appeared to affect the pharmacokinetics of dapsone. Similarly, dapsone exposures were approximately the same between the age groups of 12-15 years (N=155) and those greater than or equal to 16 years (N=253). There was no evidence of increasing systemic exposure to dapsone over the study year in these subjects.

12.4 Microbiology

In Vivo Activity: No microbiology or immunology studies were conducted during ACZONE Gel. 7.5% clinical studies.

Drug Resistance: No dapsone resistance studies were conducted during dapsone gel clinical studies. Because no such studies were done, there are no data available as to whether dapsone treatment may have resulted in decreased susceptibility of Propionibacterium acnes, an organism associated with acne, or to other antimicrobials that may be used to treat acne. Therapeutic resistance to dapsone has been reported for Mycobacterium leprae, when patients have been treated with oral dapsone.

13 Nonclinical Toxicology

13.1 Carcinogenesis, Mutagenesis, Impairment of Fertility

Dapsone was not carcinogenic to rats when orally administered for a lifetime at dose levels up to 15 mg/kg/day (approximately 340 times the systemic exposure that is associated with the MRHD of ACZONE Gel, 7.5%, based on AUC comparisons).

No evidence of potential to induce carcinogenicity was obtained in a dermal study in which dapsone gel was topically applied to Tg.AC transgenic mice for approximately 26 weeks. Dapsone concentrations of 3%, 5%, and 10% were evaluated; 3% material was judged to be the maximum tolerated dosage.

Topical gels that contained dapsone at concentrations up to 5% did not increase the rate of formation of ultraviolet light-induced skin tumors when topically applied to hairless mice in a 12-month photocarcinogenicity study.

Dapsone was not mutagenic in a bacterial reverse mutation assay (Ames test) using S. typhimurium and E. coli, with and without metabolic activation, and was negative in a micronucleus assay conducted in mice. Dapsone increased both numerical and structural aberrations in a chromosome aberration assay conducted with Chinese hamster ovary (CHO) cells.

The effects of dapsone on fertility and general reproduction performance were assessed in male and female rats following oral (gavage) dosing. Dapsone reduced sperm motility at dosages of 3 mg/kg/day or greater (approximately 22 times the systemic exposure that is associated with the MRHD of ACZONE Gel, 7.5%, based on AUC comparisons). The mean numbers of embryo implantations and viable embryos were significantly reduced in untreated females mated with males that had been dosed at 12 mg/kg/day or greater (approximately 187 times the systemic exposure that is associated with the MRHD of ACZONE Gel, 7.5%, based on AUC comparisons), presumably due to reduced numbers or effectiveness of sperm, indicating impairment of fertility. Dapsone had no effect on male fertility at dosages of 2 mg/kg/day or less (approximately 15 times the systemic exposure that is associated with the MRHD of ACZONE Gel, 7.5%, based on AUC comparisons). When administered to female rats at a dosage of 75 mg/kg/day (approximately 1400 times the systemic exposure that is associated with the MRHD of ACZONE Gel, 7.5%, based on AUC comparisons) for 15 days prior to mating and for 17 days thereafter, dapsone reduced the mean number of implantations, increased the mean early resorption rate, and reduced the mean litter size. These effects were probably secondary to maternal toxicity.

Dapsone was assessed for effects on perinatal/postnatal pup development and postnatal maternal behavior and function in a study in which dapsone was orally administered to female rats daily beginning on the seventh day of gestation and continuing until the twenty-seventh day postpartum. Maternal toxicity (decreased body weight and food consumption) and developmental effects (increase in stillborn pups and decreased pup weight) were seen at a dapsone dose of 30 mg/kg/day (approximately 560 times the systemic exposure that is associated with the MRHD of ACZONE Gel, 7.5%, based on AUC comparisons). No effects were observed on the viability, physical development, behavior, learning ability, or reproductive function of surviving pups.

14 Clinical Studies

The safety and efficacy of once daily use of ACZONE Gel, 7.5%, was assessed in two 12-week multicenter, randomized, double-blind, vehicle-controlled studies. Efficacy was assessed in a total of 4340 subjects 12 years of age and older. The majority of the subjects had moderate acne vulgaris, 20 to 50 inflammatory and 30 to 100 non-inflammatory lesions at baseline, who were randomized to receive either ACZONE Gel, 7.5% or vehicle.

Treatment response was defined at Week 12 as the proportion of subjects who were rated “none” or “minimal” with at least a two-grade improvement from baseline on the Global Acne Assessment Score (GAAS), and mean absolute change from baseline in both inflammatory and non-inflammatory lesion counts. A GAAS score of “none” corresponded to no evidence of facial acne vulgaris. A GAAS score of “minimal” corresponded to a few non-inflammatory lesions (comedones) being present and to a few inflammatory lesions (papules/pustules) that may be present.

The GAAS success rate, mean reduction, and percent reduction in acne lesion counts from baseline after 12 weeks of treatment are presented in the following table.

TABLE 3
Clinical Efficacy of ACZONE ® Gel at Week 12 in Subjects with
Acne Vulgaris

| Trial 1 | | Trial 2 | |
|---|---|---|---|
| ACZONE ® | | ACZONE ® | |
| Gel, 7.5% | Vehicle | Gel, 7.5% | Vehicle |
| (N = 1044) | (N = 1058) | (N = 1118) | (N = 1120) |

Global Acne Assessment Score

| | | | | |
|---|---|---|---|---|
| GAAS Success | 30% | 21% | 30% | 21% |
| (Score 0 or 1) | | | | |

Inflammatory Lesions

| | | | | |
|---|---|---|---|---|
| Mean absolute | 16.1 | 14.3 | 15.6 | 14.0 |
| reduction | | | | |
| Mean percent | 56% | 49% | 54% | 48% |
| reduction | | | | |

Non-inflammatory Lesions

| | | | | |
|---|---|---|---|---|
| Mean absolute | 20.7 | 18.0 | 20.8 | 18.7 |
| reduction | | | | |
| Mean percent | 45% | 39% | 46% | 41% |
| reduction | | | | |

16 How Supplied/Storage and Handling

ACZONE Gel is an off-white to yellow gel with suspended particles. It is supplied in an airless pump containing a polypropylene bottle with a high density polyethylene piston.

ACZONE (dapsone) Gel, 7.5%, is supplied in the following sizes:

| | |
|---|---|
| NDC 0023-5206-30 | 30 gram pump |
| NDC 0023-5206-60 | 60 gram pump |
| NDC 0023-5206-90 | 90 gram pump |

Storage: Store at 20° C.-25° C. (68° F.-77° F.), excursions permitted to 15° C.-30° C. (59° F.-86° F.) [see USP Controlled Room Temperature]. Protect from freezing.

17 Patient Counseling Information

Advise the patient to read the FDA-approved patient labeling (Patient Information).

Hematological Effects
Inform patients that methemoglobinemia can occur with topical dapsone treatment. Advise patients to seek immediate medical attention if they develop cyanosis [see Warnings and Precautions (5.1)].
Inform patients who have G6PD deficiency that hemolytic anemia may occur with topical dapsone treatment. Advise patients to seek medical attention if they develop signs and symptoms suggestive of hemolytic anemia [see Warnings and Precautions (5.1)].

Important Administration Instructions
Advise patients to apply ACZONE Gel, 7.5%, once daily to the entire face [see Dosage and Administration (2)].
ACZONE Gel, 7.5% is for topical use only.
Do not apply ACZONE Gel, 7.5% to eyes, mouth, or mucous membranes.

Patient Information
ACZONE ® (AK-zōn) (dapsone) Gel, 7.5%

Important: For use on skin only (topical use). Do not use ACZONE Gel, 7.5% in your
mouth, eyes, or vagina.
What is ACZONE Gel, 7.5%?
ACZONE Gel, 7.5%, is a prescription medicine used on the skin (topical) to treat acne in
people 12 years and older.
ACZONE Gel, 7.5%, has not been studied in children under 12 years of age.
Before you use ACZONE Gel, 7.5%, tell your doctor about all of your medical
conditions, including if you:
have a glucose-6-phosphate dehydrogenase deficiency (G6PD)
have higher than normal levels of methemoglobin in your blood (methemoglobinentia)
are pregnant or plan to become pregnant. It is not known if ACZONE Gel, 7.5% will
harm your unborn baby.
are breastfeeding or plan to breastfeed. ACZONE Gel, 7.5% can pass into your breast
milk and may harm your baby. You and your doctor should decide if you will use
ACZONE Gel, 7.5%, or breastfeed. You should not do both.
Tell your doctor about all the medicines you take, including prescription and over-the-
counter medicines, vitamins, and herbal supplements. Especially, tell your doctor if you are
using acne medicines that contain benzoyl peroxide. Use of benzoyl peroxide with
ACZONE Gel, 7.5% at the same time may cause your skin or facial hair to temporarily
turn yellow or orange at the site of application.
How do I use ACZONE Gel, 7.5%?
Use ACZONE Gel, 7.5% exactly as your doctor tells you to use it,
Apply ACZONE Gel, 7.5% one time a day.
Gently wash and pat dry the areas of your skin where you will apply ACZONE Gel,
7.5%.
Apply a pea-sized amount of ACZONE Gel, 7.5% in a thin layer to the entire face. A
thin layer may also be applied to other affected areas as instructed by your doctor.
Rub ACZONE Gel, 7.5% in gently and completely.
Wash your hands after applying ACZONE Gel, 7.5%..
If your acne does not get better after using ACZONE Gel, 7.5% for 12 weeks, talk to
your doctor about continuing treatment.
What are the possible side effects of ACZONE Gel, 7.5%?
ACZONE Gel, 7.5% may cause serious side effects, including:
Decrease of oxygen in your blood caused by a certain type of abnormal red blood
cell (methemoglobinemia). Stop using ACZONE Gel, 7.5% and get medical help right
away if your lips, nail beds, or the inside of your mouth turns grey or blue.
Breakdown of red blood cells (hemolytic anemia). Some people with G6PD
deficiency using ACZONE Gel, 7.5% may develop mild hemolytic anemia. Stop using
ACZONE Gel, 7.5% and tell your doctor right away if you get any of the following
signs and symptoms:

| | | |
|---|---|---|
| back pain | shortness of breath | tiredness or weakness |
| dark brown urine | fever | yellow or pale skin |

The most common side effects of ACZONE Gel, 7.5% include dryness and itching of the
skin being treated.
These are not all of the possible side effects of ACZONE Gel, 7.5%. Call your doctor for
medical advice about side effects. You may report side effects to FDA at 1-800-FDA-1088.
How should I store ACZONE Gel, 7.5%?
Store ACZONE Gel, 7.5%, at room temperature 68° F. to 77° F. (20° C. to 25° C.).
Protect ACZONE Gel, 7.5% from freezing.
Keep ACZONE Gel, 7.5% and all medicines out of the reach of children.
General information about the safe and effective use of ACZONE Gel, 7.5%.
Medicines are sometimes prescribed for purposes other than those listed in a Patient
Information leaflet. Do not use ACZONE Gel, 7.5% for a condition for which it was not
prescribed. Do not give ACZONE Gel, 7.5% to other people, even if they have the same
symptoms you have. It may harm them. You can ask your doctor or pharmacist for
information about ACZONE Gel, 7.5% that is written for health professionals.
What are the ingredients in ACZONE Gel, 7.5%?
Active ingredient: dapsone
Inactive ingredients: diethylene glycol monoethyl ether, methylparaben, acrylamide/sodium
acryloyldimethyl taurate copolymer, isohexadecane, polysorbate 80, and purified water.

What is claimed is:

1. A method of treating acne vulgaris in a subject in need thereof, the method comprising:
administering a topical pharmaceutical gel composition comprising about 7.5% w/w dapsone to the entire face of the subject at a frequency of once a day for at least 4 weeks;
wherein the method is therapeutically effective to reduce the number of lesions on the face of the subject; and
wherein the topical pharmaceutical gel composition does not comprise adapalene.

2. The method of claim 1, wherein the topical pharmaceutical gel composition is administered for at least 8 weeks.

3. The method of claim 1, wherein the topical pharmaceutical gel composition is administered for at least 12 weeks.

4. The method of claim 3, wherein the lesions comprise inflammatory lesions.

5. The method of claim 4, wherein the method is effective o reduce inflammatory lesions by 56%.

6. The method of claim 3, wherein the lesions comprise non-inflammatory lesions.

7. The method of claim 6, wherein the method is effective to reduce non-inflammatory lesions by 45%.

8. The method of claim 3, wherein the method is effective to reduce the amount of local cutaneous irritation in the subject over the treatment duration.

9. The method of claim 8, wherein the local cutaneous irritation comprises erythema.

10. The method of claim 8, wherein the local cutaneous irritation comprises scaling.

11. The method of claim 8, wherein the local cutaneous irritation comprises dryness.

12. The method of claim 8, wherein the local cutaneous irritation comprises stinging/burning.
